(19) United States
(12) Patent Application Publication
(10) Pub. No.: US 2021/0001121 A1
(43) Pub. Date: Jan. 7, 2021

(54) MUSCLE STIMULATION APPARATUS, MUSCLE STIMULATION PAD, MUSCLE STIMULATION SYSTEM, AND METHOD OF STIMULATING MUSCLES USING THE SAME

(71) Applicant: Ationlab Inc., Suwon-si (KR)

(72) Inventors: Ho Bin KIM, Anyang-si (KR); Wonseok CHOI, Anyang-si (KR)

(73) Assignee: Ationlab Inc., Suwon-si (KR)

(21) Appl. No.: 16/522,068

(22) Filed: Jul. 25, 2019

(30) Foreign Application Priority Data
Jul. 5, 2019 (KR) 10-2019-0081011

(51) Int. Cl. A61N 1/36; A61N 1/04; A61B 5/053; A61B 5/0205; A61B 5/0402; A61B 5/11

(52) U.S. Cl. CPC A61N 1/36031; A61N 1/0452; A61N 1/0492; A61N 1/36034; A61B 5/024; A61B 5/0205; A61B 5/0402; A61B 5/1107; A61B 5/0531

(57) ABSTRACT

Disclosed is a muscle stimulation system including: a muscle stimulation apparatus configured to determine a frequency corresponding to user's body information on the basis of the user's body information and determine an electrical stimulation waveform corresponding to the determined frequency; and a muscle stimulation pad which is connected to the muscle stimulation apparatus, is attached to the user's body, and includes at least one electrode configured to stimulate the user's body according to the determined electrical stimulation waveform. The muscle stimulation system may provide an electrical stimulation corresponding to the user's body information.

### CROSS-REFERENCE TO RELATED APPLICATION

This application claims priority to Korean Patent Application No. 10-2019-0081011, filed on Jul. 5, 2019, the disclosure of which is incorporated herein by reference in its entirety.

### BACKGROUND

The present disclosure relates to a muscle stimulation apparatus, a muscle stimulation system, and a method of stimulating muscles using the same.

In recent years, mechanical vibratory massage therapy and low-frequency therapeutic apparatuses have been generally used to help rehabilitate muscles. Vibratory massage therapy is a therapy that uses mechanical vibration for therapeutic purposes, and it transmits vibrations to the body to create a longitudinal wavy flow, which improves blood flow and relaxes contracted muscle tissue, thereby helping to rehabilitate damaged muscle tissue. It can achieve pain relief, lipid degradation and cosmetic effects, increased blood circulation, congestion mitigation, and muscle relaxation.

A low-frequency therapeutic apparatus is an apparatus that is used to treat nerve or muscle damage by applying low-frequency pulses to the living body. It is used for various purposes, such as fatigue recovery, physical therapy, obesity treatment, or pain relief.

In the past, the vibratory massage apparatus or low-frequency therapeutic apparatus as described above could be used in a hospital or a rehabilitation center. However, with the recent development of technology, various types of muscle vibration massage apparatuses have been commercialized, which may be used at home and are miniaturized to be portable and also have improved performance.

However, the conventional muscle vibration massage apparatus and conventional low-frequency therapeutic apparatus as described above have a problem in that they provide only a standardized stimulation without considering the physical characteristics that are different from person to person. In addition, a problem also arises in that providing such a standardized stimulation causes unnecessary pain to the user.

In order to solve such problems, a conventional muscle vibration massage apparatus uses a method of manually adjusting the intensity of vibration, and a conventional low-frequency therapy apparatus uses a method such as manually adjusting the intensity of electrical stimulation. However, these methods merely adjust the intensity of the stimulation only, and still have the problem that they do not provide a suitable stimulation to the user.

### PRIOR ART DOCUMENTS

### Patent Documents

Korean Patent Application Laid-Open Publication No. 10-2017-0127581 (Nov. 22, 2017).

### SUMMARY

An object of the present disclosure is to provide a muscle stimulation apparatus, a muscle stimulation system and a method of stimulating muscles using the same, which can acquire the user's body information by measuring the impedance value of the user, determine an electrical stimulation waveform corresponding to the user on the basis of the user's body information, provide the most suitable electrical stimulation to the user by modulating the electrical stimulation waveform according to the user's surrounding environment and body-specific information, and enable the user himself/herself to receive an electrical stimulation without specialist expertise.

Objects which are to be achieved by the present disclosure are not limited to the above-mentioned object, and other objects which are not mentioned will be clearly understood by those skilled in the art from the following description.

The present disclosure provides a muscle stimulation system including: a muscle stimulation apparatus configured to determine a frequency corresponding to user's body information on the basis of the user's body information, and determine an electrical stimulation waveform corresponding to the determined frequency; and a muscle stimulation pad which is connected to the muscle stimulation apparatus, is attached to the user's body, and includes at least one electrode configured to stimulate the user's body according to the determined electrical stimulation waveform.

In one embodiment, the muscle stimulation apparatus may be configured to operate the muscle stimulation pad using a plurality of predetermined electrical stimulation waveforms and acquire the user's body information using the user's impedance value measured by operating the muscle stimulation pad.

In another embodiment, the muscle stimulation pad may include at least one of an electrocardiogram sensor, a breath sensor, a pulse rate sensor, a weight sensor, a gyro sensor, an accelerometer sensor, a flex sensor, and a shock sensor.

In still another embodiment, the sensor included in the muscle stimulation pad may be used to measure the user's impedance value or measure at least one of the user's posture and motion.

In yet another embodiment, the muscle stimulation system may further include: a user terminal configured to receive the user's body information; and a management sensor configured to receive the user's body information from the user terminal and identify the electrical stimulation waveform corresponding to the user's body information.

In still yet another embodiment, the muscle stimulation apparatus may be configured to amplify or modulate the electrical stimulation waveform according to any one of the user's surrounding environment and body-specific information.

The present disclosure also provides a muscle stimulation apparatus including: a body information acquisition unit configured to acquire user's body information; a waveform determination unit configured to determine a frequency corresponding to the users' body information on the basis of the acquired user's body information and determine an electrical stimulation waveform corresponding to the frequency; and a control unit configured to control the muscle stimulation pad according to the electrical stimulation waveform determined in the waveform determination unit.

In one embodiment, the control unit may be configured to operate the muscle stimulation pad using a plurality of predetermined electrical stimulation waveforms, and the body information acquisition unit may be configured to acquire the user's body information using a user's impedance value measured by operating the muscle stimulation pad.

In another embodiment, the muscle stimulation apparatus may include at least one of an electrocardiogram sensor, a breath sensor, a pulse rate sensor, a weight sensor, a gyro sensor, an accelerometer sensor, a flex sensor, and a shock sensor.

In still another embodiment, the sensor included in the muscle stimulation pad may be used to measure the user's impedance value and or measure at least one of the user's posture and motion.

In yet another embodiment, the muscle stimulation apparatus may further include a communication unit configured to receive an electrical stimulation waveform corresponding to the user's body information from a management server.

In still yet another embodiment, the muscle stimulation apparatus may further include a waveform modulation unit configured to amplify or modulate the electrical stimulation waveform according to any one of the user's surrounding environment and body-specific information.

The present disclosure also includes a method for stimulating muscles, including: acquiring body information by a muscle stimulation apparatus; determining a frequency corresponding to the acquired body information by the muscle stimulation apparatus; determining an electrical stimulation waveform corresponding to the determined frequency by the muscle stimulation apparatus; and operating a muscle stimulation pad attached to the user's body by the muscle stimulation apparatus.

In one embodiment, the determining of the frequency corresponding to the acquired body information comprises receiving an electrical stimulation waveform corresponding to the user's body information from a management server by the muscle stimulation apparatus.

In another embodiment, the determining of the electrical stimulation waveform comprises amplifying or modulating the electrical stimulation waveform according to any one of the user's surrounding environment and body-specific information by the muscle stimulation apparatus.

The present disclosure also provides an apparatus including: one or more processors; a memory; and one or more programs, wherein the one or more programs are stored in the memory and configured to be executed by the one or more processors, and wherein the programs are configured to perform: an operation of causing the apparatus to acquire body information; an operation of determining a frequency corresponding to the acquired body information; an operation of determining an electrical stimulation waveform corresponding to the determined frequency; and an operation of operating a muscle stimulation pad attached to the users' body.

The present disclosure also provides a muscle stimulation pad which is connected to a muscle stimulation apparatus, is attached to a user's body, and includes at least one electrode assembly configured to stimulate the user's body according to an electrical stimulation waveform corresponding to the user's body information received from the muscle stimulation apparatus.

In one embodiment, the electrode assembly may include a positive electrode, a negative electrode, and a wire, wherein the positive electrode and the negative electrode may be isolated from each other by an isolating material.

In another embodiment, the muscle stimulation pad may at least one of an electrocardiogram sensor, a breath sensor, a pulse rate sensor, a weight sensor, a gyro sensor, an accelerometer sensor, a flex sensor, and a shock sensor.

In still another embodiment, the electrical stimulation waveform may be a waveform obtained by determining a frequency corresponding to user's body information on the basis of the user's body information and determining a waveform corresponding to the determined frequency.

### BRIEF DESCRIPTION OF THE DRAWINGS

FIG. 1 is a diagram showing the configuration of a muscle stimulation system according to an embodiment of the present disclosure.

FIG. 2 is a block diagram showing the configuration of a muscle stimulation apparatus according to an embodiment of the present disclosure.

FIG. 3 is a flow chart showing a muscle stimulation method according to an embodiment of the present disclosure.

FIG. 4 is a block diagram showing an exemplary computing environment including an exemplary computing apparatus suitable for use in an embodiment of the present disclosure.

### DETAILED DESCRIPTION

Hereinafter, exemplary embodiments of the present disclosure will be described with reference to the accompanying drawings. However, these embodiments are illustrative only and the scope of the present disclosure is not limited thereto.

In the following description, when the detailed description of known technology related to the present disclosure is determined to unnecessarily obscure the subject matter of the present invention, it will be omitted. In addition, the terms used below are defined considering their functions in the present disclosure, and can be changed depending upon the intentions or practices of a user or an operator, etc. Accordingly, the definition thereof should be determined based on the contents throughout this specification.

The technical spirit of the present disclosure is determined by the appended claims, and the following embodiments are merely means for efficiently explaining the technical spirit of the present disclosure to those skilled in the art to which the present disclosure pertains.

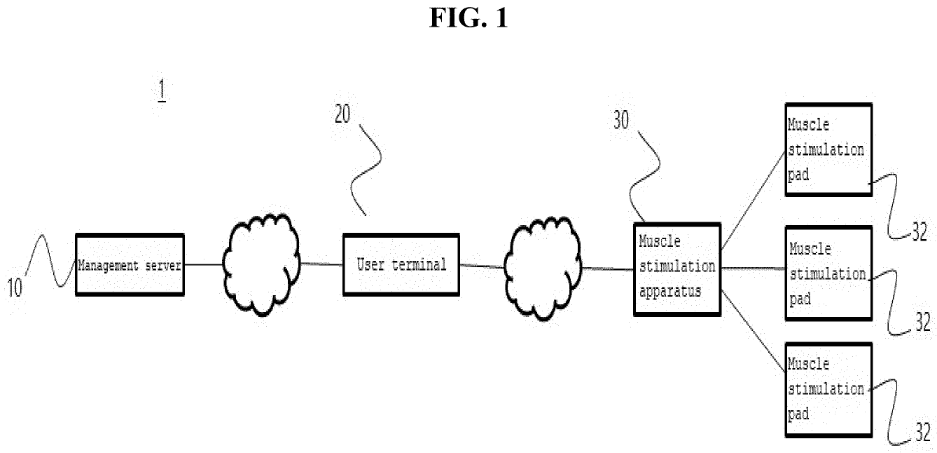

FIG. 1 is a diagram showing the configuration of a muscle stimulation system 1 according to an embodiment of the present disclosure.

As shown therein, the muscle stimulation system 1 according to an embodiment of the present disclosure includes a management server 10, a user terminal 20, a muscle stimulation apparatus 30, and at least one muscle stimulation pad 32.

The management server 10 is connected to the user terminal 20 and the muscle stimulation apparatus 30 so as to be communicable with each other via a network such as a local area network (LAN), a wide area network (WAN), a cellular network, or the Internet. Examples of the user terminal 20 include, but are not limited to, smart phones, mobile phones, PDAs (Personal Digital Assistants), MP3, tablet PCs, PMP (Portable Multimedia Players), laptop computers, personal computers, external body composition analyzers, etc., as well as various types of wireless communication devices and wired communication devices that may be connected to the management server 10.

The management serve 10 may store and manage an electrical stimulation waveform corresponding to user's body information. Specifically, the management server 10 may store electrical stimulation waveform data corresponding to user's body information, receive the user's body information from the user terminal 20, and identify an electrical stimulation waveform corresponding to the user's body. Alternatively, the management server 10 may also calculate an electrical stimulation waveform corresponding to the user's body. For example, the management server 10 may calculate an optimal electrical stimulation waveform using the waveform's frequency and duty cycle value according to the user's body information.

An embodiment of the present disclosure will now be described, in which the muscle stimulation apparatus 30 described below determines a frequency corresponding to user's body information and determines an electrical waveform on the basis of the user's body information.

The user terminal 20 is a user terminal that uses the muscle stimulation apparatus 30, and may receive user's body information from the user. Specifically, the user terminal 20 may receive the user's body information and transmit it to the management server 10.

The muscle stimulation apparatus 30 may provide electrical stimulation to the user by at least one muscle stimulation pad 32. Specifically, the muscle stimulation apparatus 30 may provide electrical stimulation to the user by an electrical stimulation method using FES (Functional Electrical Stimulation), EMS (Electrical Muscle Stimulation) and TENS (Transcutaneous Electrical Nerve Stimulation).

The muscle stimulation apparatus 30 may acquire user's body information. Specifically, the muscle stimulation apparatus may operate at least one muscle stimulation pad 32 may operate at least one muscle stimulation pad 32 using a plurality of predetermined electrical stimulation waveforms and acquire user's body information using a user's impedance value measured by measuring the muscle stimulation pad 32. Here, the impedance value may refer to, for example, the user's electrocardiogram, the user's posture, position, etc. In addition, the body information refers to information about the user's body part to which the muscle stimulation pad 32 is being attached. Even if the user is the same person, if a different impedance value is identified according to the attachment position of the muscle stimulation pad 32, body information according thereto may be acquired.

The muscle stimulation apparatus 30 may be provided with a sensor for measuring the user's impedance value. Specifically, the muscle stimulation apparatus 30 may include an electrocardiogram sensor, a breath sensor and a pulse rate sensor, which are used to measure the user's electrocardiogram, breath and pulse rate, respectively. In addition, the muscle stimulation sensor 30 may be provided with at least one of a weight sensor, a gyro sensor, an accelerometer sensor, a flex sensor, and a shock sensor, which is used to measure the user's posture or motion. Namely, the muscle stimulation apparatus 30 may use the sensor included therein to measure the user's impedance value and to estimate the user's posture or motion. For example, the muscle stimulation apparatus 30 may use a weight sensor to measure the user's body weight. Although an embodiment of the present disclosure describes that the muscle stimulation apparatus 30 acquires user's body information by measuring the impedance value, it is to be understood that the scope of the present disclosure is not limited thereto and the muscle stimulation apparatus 30 may receive user's body information from the management sever 10. It is described herein that a sensor is provided in the muscle stimulation apparatus 30, but it is to be understood that the scope of the present disclosure is not limited thereto and the sensor may also be provided in the muscle stimulation pad 32 connected to the muscle stimulation apparatus 30.

The muscle stimulation apparatus 30 may determine an electrical stimulation waveform corresponding to the user's body information. Specifically, the muscle stimulation apparatus 30 may determine a frequency corresponding to the user's body information on the basis of the user's body information and determine an electrical stimulation waveform corresponding to the determined frequency. The muscle stimulation apparatus 30 may provide the determined electrical stimulation waveform to the user through at least one muscle stimulation pad 32.

Since the muscle stimulation apparatus 30 determines an electrical stimulation waveform corresponding to the user's body information, it makes it possible to provide a suitable electrical stimulation corresponding to the user's physical characteristics and does not cause unnecessary pain to the user.

The muscle stimulation pad 32 may provide electrical stimulation corresponding to the input of the muscle stimulation apparatus 30 to the user. Specifically, the muscle stimulation pad 32 may be connected by wire or wireless to the muscle stimulation apparatus 30, may be attached to or worn by the user's body, and may provide an electrical stimulation, which corresponds to an electrical stimulation waveform determined in the muscle stimulation apparatus 30, to the user's body.

The muscle stimulation pad 32 may include one or more electrode assemblies configured to apply electrical stimulation to the user's body. Here, the electrode assembly may include a positive electrode, a negative electrode a positive electrode, a negative electrode, a wire, and an insulating material for separating the positive electrode from the negative electrode.

The muscle stimulation pads 32 may be formed in a plurality of shapes according to the user's body part and may be attached to the user's body. For example, the muscle stimulation pad 32 for attachment to the user's back may be formed of a plate-shaped wide sheet, and the muscle stimulation pad 32 for wearing on the user's hand or foot may be formed in the shape of a shoe or a glove.

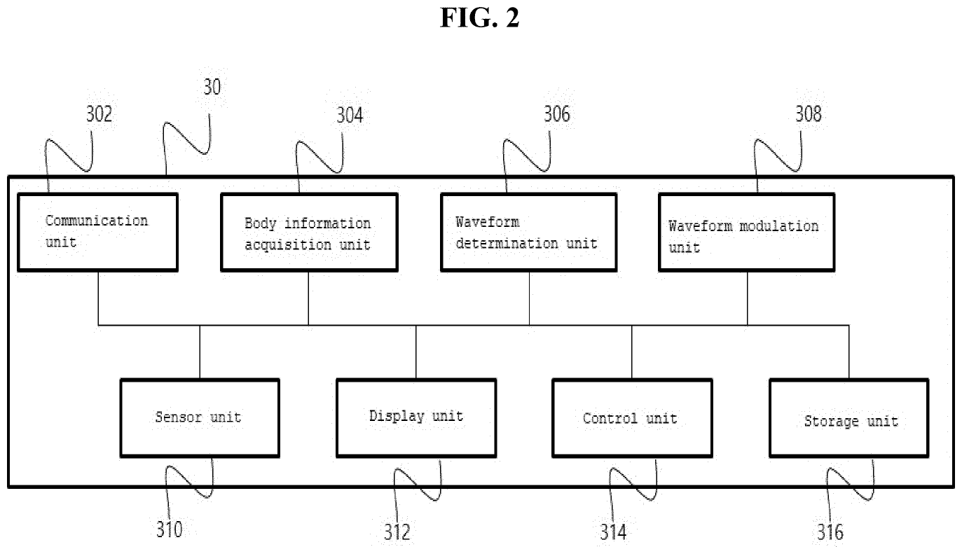

FIG. 2 is a block diagram showing the configuration of a muscle stimulation apparatus 30 according to an embodiment of the present disclosure.

As shown therein, the muscle stimulation apparatus 30 according to an embodiment includes a communication unit 302, a body information acquisition unit 304, a waveform determination unit 306, a waveform modulation unit 308, a sensor unit 310, a display unit 312, a control unit 314, and a storage unit 316.

The communication unit 302 may communicate with the management sever 10. Specifically, the communication unit 302 may communicate with the management server 10 via a network, such as a local area network (LAN), a wide area network (WAN), a cellular network, or the Internet.

In addition, the communication unit 302 may communicate with the muscle stimulation pad 32. Specifically, the communication unit 302 may be connected by wire or wireless to at least one muscle stimulation pad 32 and may communicate with the muscle stimulation pad 32 so that an electrical stimulation waveform determined in the waveform determination unit 306 and the waveform modulation unit 308 may be provided to the user.

The body information acquisition unit 304 may acquire user's body information. Specifically, the body information acquisition unit 304 may acquire user's body information from the management server 10, or may acquire user's body information using the user's impedance value sensed in the sensor unit 310.

The waveform determination unit 306 may determine an electrical stimulation waveform corresponding to the user's body information. Specifically, the waveform determination unit 306 may determine a frequency corresponding to the user's body information on the basis of the user's body information and determine an electrical stimulation waveform corresponding to the determined frequency. Here, the body information refers to information about the user's body part to which the muscle stimulation pad 32 is being attached. Even if the user is the same person, when a different impedance value is identified according to the attachment position of the muscle stimulation pad 32, body information according thereto may be acquired.

The waveform modulation unit 308 may modulate the electrical stimulation waveform determined in the waveform determination unit 306. Specifically, the waveform modulation unit 308 may amplify or modulate the determined electrical stimulation waveform according to the user's surrounding environment and body-specific information. The waveform modulation unit 308 may amplify or modulate the electrical stimulation waveform so that a sensation (e.g., thermal sensation, chill, pain, etc.) to be provided to the user through the electrical stimulation can be accurately delivered.

Namely, the muscle stimulation apparatus 30 can acquire user's body information using the user's impedance value, determine an electrical stimulation waveform on the basis of the user's body information, provide the most suitable electrical stimulation to the user by modulating the electrical stimulation waveform according to the user's surrounding environment and body-specific information, and enable the user himself/herself to receive an electrical stimulation without specialist expertise.

The sensor unit 310 may measure the user's impedance value. Specifically, the sensor unit 310 may include an impedance sensor for measuring the user's impedance value. For example, the impedance sensor may include a breath sensor for measuring the user's breath using the user's breathing signal, a pulse rate sensor capable of measuring the user's pulse rate using the user's pulse signal, etc. Although representative examples for measurement of the user's impedance value are merely described here, it is to be understood that the scope of the present disclosure is not limited thereto and the sensor unit 310 may include various sensors for measuring the user's impedance value.

The sensor unit 310 may measure the user's posture or motion. Specifically, the sensor unit 310 may include at least one of a weight sensor, a gyro sensor, an accelerometer sensor, a flex sensor, and a shock sensor in order to sense one or more of the user's posture and motion. Although representative examples of sensors for measuring the user's posture or motion are merely described here, it is to be understood that the scope of the present disclosure is not limited thereto and the sensor unit 310 may include various sensors for measuring the user's posture or motion.

The display unit 312 can visually present the current situation to the user. The display unit 312 may be provided on one side of the muscle stimulation apparatus 30, and can display the electrical stimulation waveform being provided to the user or can visually present the user's body information. Although a representative example that is provided to the user by the display unit 312 is described herein, it is to be understood that the scope of the present disclosure is not limited thereto and the display unit 312 may display all information that can be provided to the user.

The control unit 314 can control the muscle stimulation apparatus 30 according to a user's input. Specifically, the control unit 314 can turn on/off the apparatus according to a user's input or adjust the electrical stimulation intensity. To this end, an input unit that can be input by the user may be provided on one side of the muscle stimulation apparatus 30.

The storage unit 316 may store various information used in the muscle stimulation apparatus 30. Specifically, the storage unit 316 may store the user's impedance value, the user's body information, the electrical stimulation waveform which was recently used by the user, etc. Although representative examples that are stored in the storage unit 316 are merely described here, it is to be understood that the scope of the present disclosure is not limited thereto and the storage unit 316 may store all information that can be stored in the muscle stimulation apparatus 30.

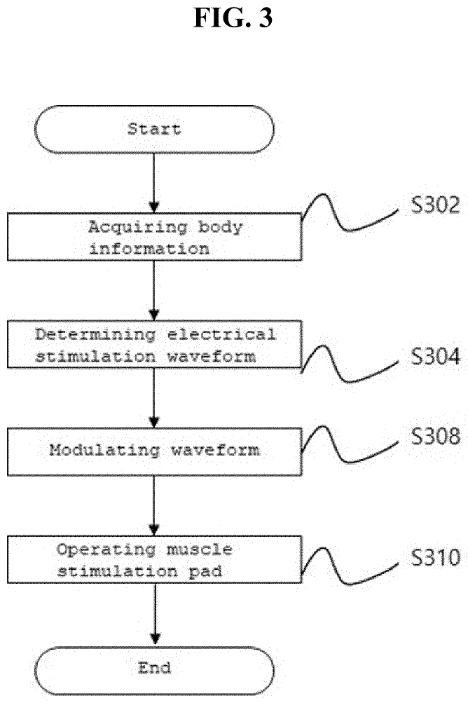

FIG. 3 is a flow chart showing a muscle stimulation method according to an embodiment of the present disclosure. Although FIG. 3 shows that the method includes a plurality of divided steps, at least some of the steps may be performed in a different order, or may be performed in combination with other steps, or may be omitted, or may be divided into sub-steps, or one or more steps not shown in the figure may additionally be performed. In addition, in some embodiments, one or more steps not shown in the figure may be performed with the method.

The muscle stimulation apparatus 30 acquires user's body information (S302). Specifically, the muscle stimulation apparatus 30 may acquire user's body information from the management server 10, or may acquire user's body information using the user's impedance value sensed in the sensor unit 310.

Next, the muscle stimulation apparatus 30 determines an electrical stimulation waveform (S304). Specifically, the muscle stimulation apparatus 30 may determine a frequency corresponding to the user's body information on the basis of the user's body information, and determine an electrical stimulation waveform corresponding to the determined frequency.

Next, the muscle stimulation apparatus 30 modulates the determined electrical stimulation waveform (S308). Specifically, the muscle stimulation apparatus 30 may amplify or modulate the determined electrical stimulation waveform according to the user's surrounding environment and body-specific information.

Next, the muscle stimulation apparatus 30 operates the muscle stimulation pad 32 (S310). Specifically, the muscle stimulation apparatus 30 may enable the muscle stimulation pad to be operated according to the determined electrical stimulation waveform or the modulated electrical stimulation waveform.

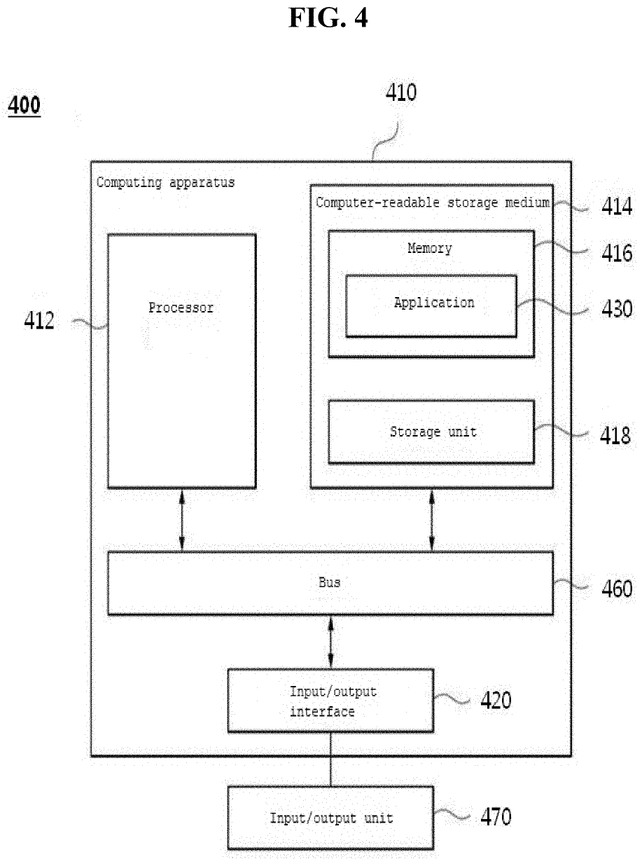

FIG. 4 illustrates an exemplary computing environment including an exemplary computing apparatus suitable for use in exemplary embodiments.

An exemplary computing environment 400 illustrated in FIG. 4 includes a computing apparatus 410. Generally, each element may have a different function and ability, and may additionally include a component suitable for the element, although not shown below. The computing apparatus may be an apparatus for stimulating muscles (e.g., the muscle stimulation apparatus 30), or an apparatus configured to receive body information (e.g., the user terminal 20), or an apparatus configured to acquire body information or store a waveform (e.g., the server 20).

The computing apparatus 410 includes at least one processor 412, computer-readable storage medium 414 and bus 460. The processor 412 is connected to the bus 460, and the bus 460 connects the processor 412 to other various components of the computing apparatus 410, including the computer-readable storage medium 414.

The processor 412 enables the computing apparatus 410 to be operated according to the above-mentioned exemplary embodiment. For example, the processor 412 can execute computer-executable instructions stored in the computer-readable storage medium 414, and when the computer-executable instructions stored in the computer-readable storage medium 414 are executed by the processor 412, they may be configured to enable the computing apparatus 410 to perform operations according to a specific exemplary embodiment.

The computer-readable storage medium 414 is configured to store computer-executable instructions or program codes (e.g., instructions included in an application 430), program data (e.g., data used by the application 430), and/or other suitable forms of information. The application 430 stored in the computer-readable storage medium 414 includes a predetermined set of instructions executable by the processor 412.

The memory 416 and storage unit 418 shown in FIG. 4 are examples of the computer-readable storage medium 414. In the memory 416, computer-executable instructions executable by the processor 412 may be loaded. In addition, program data may be stored in the memory 416. For example, this memory 416 may be volatile memory such as random access memory, nonvolatile memory, or a suitable combination thereof. As another example, the storage unit 418 may include one or more removable or non-removable components for storage of information. For example, the storage unit 418 may be a hard disk, a flask memory, a magnetic disk, an optical disk, other form of storage medium that can be accessed by the computing apparatus 410 and can store desired information, or a suitable combination thereof.

The computing apparatus 410 may also include one or more input/output interfaces 420 that provide interfaces for one or more input/output units 470. The input/output interfaces 420 are connected to the bus 460. The input/output units 470 may be connected to other components of the computing apparatus 410 through the input/output interfaces 420. The input/output units 470 may include input devices such as a pointing device, a keyboard, a touch input device, a voice input device, a sensor device and/or a photographing device, and/or output devices such as a display device, a printer, a speaker and/or a network card.

As described above, according to the present disclosure, a muscle stimulation apparatus, a muscle stimulation system and a method of stimulating muscles using the same are provided, which can acquire user's body information by measuring the impedance value of the user, determine an electrical stimulation waveform corresponding to the user on the basis of the user's body information, provide the most suitable electrical stimulation to the user by modulating the electrical stimulation waveform according to the user's surrounding environment and body-specific information, and enable the user himself/herself to receive an electrical stimulation without specialist expertise.

While the exemplary embodiments of the present disclosure have been described in detail, those skilled in the art will understand that various modifications to the above-described embodiments are possible without departing from the scope of the present disclosure. Therefore, the scope of the present invention should not be limited to the described embodiments, but should be defined by the appended claims and equivalents thereof.

What is claimed is:

1. A muscle stimulation system comprising:
a muscle stimulation apparatus configured to determine a frequency corresponding to user's body information on the basis of the user's body information and determine an electrical stimulation waveform corresponding to the determined frequency; and
a muscle stimulation pad which is connected to the muscle stimulation apparatus, is attached to the user's body, and comprises at least one electrode configured to stimulate the user's body according to the determined electrical stimulation waveform.

2. The muscle stimulation system of claim 1, wherein the muscle stimulation apparatus is configured to operate the muscle stimulation pad using a plurality of predetermined electrical stimulation waveforms and acquire the user's body information using a user's impedance value measured by operating the muscle stimulation pad.

3. The muscle stimulation system of claim 2, wherein the muscle stimulation pad comprises at least one of an electrocardiogram sensor, a breath sensor, a pulse rate sensor, a weight sensor, a gyro sensor, an accelerometer sensor, a flex sensor, and a shock sensor.

4. The muscle stimulation system of claim 3, wherein the muscle stimulation apparatus is configured to measure the user's impedance value or at least one of user's posture or motion by the sensor of the muscle stimulation pad.

5. The muscle stimulation system of claim 1, further comprising:
a user terminal configured to receive the user's body information; and
a management sensor configured to receive the user's body information from the user terminal and identify the electrical stimulation waveform corresponding to the user's body information.

6. The muscle stimulation system of claim 1, wherein the muscle stimulation apparatus is configured to amplify or modulate the electrical stimulation waveform according to any one of user's surrounding environment and body-specific information.

7. A muscle stimulation apparatus comprising:
a body information acquisition unit configured to acquire user's body information;
a waveform determination unit configured to determine a frequency corresponding to the users' body information on the basis of the acquired user's body information and determine an electrical stimulation waveform corresponding to the determined frequency; and
a control unit configured to control a muscle stimulation pad according to the electrical stimulation waveform determined in the waveform determination unit.

8. The muscle stimulation apparatus of claim 7, wherein the control unit is configured to operate the muscle stimulation pad using a plurality of predetermined electrical stimulation waveforms, and the body information acquisition unit is configured to acquire the user's body information using a user's impedance value measured by operating the muscle stimulation pad.

9. The muscle stimulation apparatus of claim 8, wherein the muscle stimulation apparatus comprises at least one of an electrocardiogram sensor, a breath sensor, a pulse rate sensor, a weight sensor, a gyro sensor, an accelerometer sensor, a flex sensor, and a shock sensor.

10. The muscle stimulation apparatus of claim 9, wherein the muscle stimulation apparatus is configured to measure the user's impedance value or at least one of user's posture or motion by the sensor of the muscle stimulation pad.

11. The muscle stimulation apparatus of claim 7, further comprising a communication unit configured to receive an electrical stimulation waveform corresponding to the user's body information from a management server.

12. The muscle stimulation apparatus of claim 7, further comprising a waveform modulation unit configured to amplify or modulate the electrical stimulation waveform according to any one of the user's surrounding environment and body-specific information.

13. A method for stimulating muscles, comprising:
acquiring body information by a muscle stimulation apparatus;
determining a frequency corresponding to the acquired body information by the muscle stimulation apparatus;
determining an electrical stimulation waveform corresponding to the determined frequency by the muscle stimulation apparatus; and
operating a muscle stimulation pad attached to the user's body by the muscle stimulation apparatus.

14. The method of claim 13, wherein the determining of the frequency corresponding to the acquired body information comprises receiving an electrical stimulation waveform corresponding to the user's body information from a management server by the muscle stimulation apparatus.

15. The method of claim 13, wherein the determining of the electrical stimulation waveform comprises amplifying or modulating the electrical stimulation waveform according to any one of the user's surrounding environment and body-specific information by the muscle stimulation apparatus.

16. An apparatus comprising:
one or more processors;
a memory; and
one or more programs,
wherein the one or more programs are stored in the memory and configured to be executed by the one or more processors, and
wherein the programs are configured to perform:
an operation of causing the apparatus to acquire body information;
an operation of determining a frequency corresponding to the acquired body information;
an operation of determining an electrical stimulation waveform corresponding to the determined frequency; and
an operation of operating a muscle stimulation pad attached to the users' body.

17. A muscle stimulation pad which is connected to a muscle stimulation apparatus, is attached to a user's body, and comprises at least one electrode assembly configured to stimulate the user's body according to an electrical stimulation waveform corresponding to user's body information received from the muscle stimulation apparatus.

18. The muscle stimulation pad of claim 17, wherein the electrode assembly comprises a positive electrode, a negative electrode, and a wire, wherein the positive electrode and the negative electrode are separated from each other by an isolating material.

19. The muscle stimulation pad of claim 17, wherein the muscle stimulation pad comprises at least one of an electrocardiogram sensor, a breath sensor, a pulse rate sensor, a weight sensor, a gyro sensor, an accelerometer sensor, a flex sensor, and a shock sensor.

20. The muscle stimulation pad of claim 17, wherein the electrical stimulation waveform is a waveform obtained by determining a frequency corresponding to user's body information on the basis of the user's body information and determining a waveform corresponding to the determined frequency.
